SEQUENCE LISTING (16 sequences)

SEQ ID NO: 1 - DNA, 8578 bp, Artificial Sequence
  enhancer (1)..(659): CMV IE
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataacagtgtccgcagaagcaaggggctgaaaacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacggcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaacaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcaccgacaagacctcagccacggtcatctgccgcaaaaatgccagcattagcgtgcgggcccaggaccgctactatagctcatcttggagcgaatgggcatctgtgccctgcagtccatggagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtgcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 2 - protein, 527 aa, Artificial Sequence
  Description of Artificial Sequence Plasmid
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPNNLQLKPLKNSRQVEVSWEYPDTWSTPHSYFSLTFCVQVQGKSKREKKDRVFTDKTSATVICRKNASISVRAQDRYYSSSWSEWASVPCSPWRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 3 - DNA, 8578 bp, Artificial Sequence; identical to SEQ ID NO: 1
  Description of Artificial Sequence plasmid

SEQ ID NO: 4 - protein, 526 aa, Artificial Sequence
  Description of Artificial Sequence plasmid
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPNNLQLKPLKNSRQVEVSWEYPDTWSTPHSYFSLTFCVQVQGKSKREKKDRVFTDKTSATVICRKNASISVRAQDRYYSSSWSEWASVPCSPWRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNA

SEQ ID NO: 5 - DNA, 8623 bp, Artificial Sequence
  misc_feature (1)..(750): CMV enhancer and promoter
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataacagtgtccgcagaagcaaggggctgaaaacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacggcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaacaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcaccgacaagacctcagccacggtcatctgccgcaaaaatgccagcattagcgtgcgggcccaggaccgctactatagctcatcttggagcgaatgggcatctgtgccctgcagtggtggcggtggaagcggtggcggtggaagcggtggcggtggaagcccatggagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtgcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 6 - protein, 542 aa, Artificial Sequence
  Description of Artificial Sequence plasmid
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPNNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSPWRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 7 - DNA, 8608 bp, Artificial Sequence
  Description of Artificial Sequence plasmid
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataacagtgtccgcagaagcaaggggctgaaaacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacggcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaacaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcaccgacaagacctcagccacggtcatctgccgcaaaaatgccagcattagcgtgcgggcccaggaccgctactatagctcatcttggagcgaatgggcatctgtgccctgcagtggtggcggtggaagcggtggcggtggaagcccatggagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtgcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 8 - protein, 537 aa, Artificial Sequence
  Description of Artificial Sequence plasmid
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPNNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSPWRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 9 - DNA, 8638 bp, Artificial Sequence
  Description of Artificial Sequence plasmid
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataacagtgtccgcagaagcaaggggctgaaaacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacggcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaacaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcaccgacaagacctcagccacggtcatctgccgcaaaaatgccagcattagcgtgcgggcccaggaccgctactatagctcatcttggagcgaatgggcatctgtgccctgcagtggtggcggtggaagcggtggcggtggaagcggtggcggtggaagcggtggcggtggaagcccatggagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtgcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 10 - protein, 547 aa, Artificial Sequence
  Description of Artificial Sequence plasmid
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPNNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSPWRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 11 - DNA, 8659 bp, Artificial Sequence
  misc_feature (1)..(750): CMV enhancer and promoter
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagggcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaacaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcaccgacaagacctcagccacggtcatctgccgcaaaaatgccagcattagcgtgcgggcccaggaccgctactatagctcatcttggagcgaatgggcatctgtgccctgcagtggtggcggtggaagcggtggcggtggaagcggtggcggtggaagcggtggcggtggaagcccatggagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataacagtgtccgcagaagcaaggggctgaaacggccgatcactagtgaattcgcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactttagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 12 - protein, 547 aa, Artificial Sequence; identical to SEQ ID NO: 10
  Description of Artificial Sequence plasmid

SEQ ID NO: 13 - DNA, 8644 bp, Artificial Sequence
  Description of Artificial Sequence plasmid
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagggcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaacaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcaccgacaagacctcagccacggtcatctgccgcaaaaatgccagcattagcgtgcgggcccaggaccgctactatagctcatcttggagcgaatgggcatctgtgccctgcagtggtggcggtggaagcggtggcggtggaagcggtggcggtggaagcccatggagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataacagtgtccgcagaagcaaggggctgaaacggccgatcactagtgaattcgcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 14 - protein, 542 aa, Artificial Sequence; identical to SEQ ID NO: 6
  Description of Artificial Sequence plasmid

SEQ ID NO: 15 - DNA, 8629 bp, Artificial Sequence
  Description of Artificial Sequence plasmid
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagggcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaacaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcaccgacaagacctcagccacggtcatctgccgcaaaaatgccagcattagcgtgcgggcccaggaccgctactatagctcatcttggagcgaatgggcatctgtgccctgcagtggtggcggtggaagcggtggcggtggaagcccatggagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataacagtgtccgcagaagcaaggggctgaaacggccgatcactagtgaattcgcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 16 - protein, 537 aa, Artificial Sequence; identical to SEQ ID NO: 8
  Description of Artificial Sequence plasmid

(19) United States
(12) Patent Application Publication
(10) Pub. No.: US 2002/0018767 A1
(43) Pub. Date: Feb. 14, 2002

(54) Anti-cancer cellular vaccine

(76) Inventors: See-Woo Lee, Seoul (KR); Han-Soo Kim, Seoul (KR)

Correspondence Address: LARSON & TAYLOR, PLC, 1199 NORTH FAIRFAX STREET, SUITE 900, ALEXANDRIA, VA, 22314, US

(21) Appl. No.: 09/828,825

(22) Filed: Apr. 10, 2001

(30) Foreign Application Priority Data
Jul. 27, 2000 (KR) 00-43498

(51) Int. Cl. A61K 48/00; C12N 5/08

(52) U.S. Cl. 424/93.21; 435/366

(57) ABSTRACT

A method for producing a cellular vaccine against any cancer is provided. The cancer cells transformed with an expression co-expressing modified interleukin-12 and a co-stimulatory molecule provides an effective means of cancer treatment since the cells can display characteristics of both cancer cells and antigen-presenting cells.

### FIELD OF THE INVENTION

The present invention relates generally to the field of recombinant DNA molecules. More specifically, the present invention relates to an expression vector comprising IL-12 and a co-stimulatory molecule and methods of using same in a cancer vaccine.

### BACKGROUND OF THE INVENTION

Although there is considerable evidence from scientific and clinical studies that the immune system is capable of destroying cancerous tissue, in most cases the immune system either fails to recognize the tumor or the response that is generated is too weak to be effective (Farzaneh et al., 1998, Immunol. Today 19:294). While early detection may cure tumors in many cases, once the disease becomes metastatic to distant organs, it is almost always fatal. Furthermore, the disappointing results observed with chemotherapy, radiotherapy and surgery, individually or in combination, has shifted the attention of many investigators to immunological or biological agents (Ockert et al., 1999. Immunol. Today 20:63). As such, increasing the capacity of the immune system to mediate tumor regression has been a major goal in tumor immunology. Progress towards this goal has recently been aided by the identification of immunogenic tumor antigens and by a better understanding of the mechanisms of T cell-mediated immune response and tumor escape (Boon et al., 1997, Immunol. Today 18:267; Chen, 1998, Immunol. Today 19:27).

An understanding of the mechanisms by which some animals reject tumors whereas others display progressive tumor outgrowth is gradually evolving based on an appreciation of the underlying concepts of cellular and tumor immunology. Simply put, these are that tumor cells can be eliminated by the immune system and that cellular cytotoxicity plays a major role in antitumor immunity and the effector cells in many cases are either CD8⁺ CTL or CD4⁺ Th cells (Denfeld et al., Int J. Cancer 62:259; Greten and Jaffee, 1999, J. Clin. Oncol. 17:1047; Sampson et al., 1996, Proc. Natl. Acad. Sci. USA 93:10399). However, the induction and amplification of an effective T cell-mediated immune response in malignancies characterized by poor immungenicity is the most challenging aspects of tumor vaccine development (Sampson et al., 1996). A two signal model of lymphocyte activation postulates that for optimal activation, lymphocytes require both an antigen-specific signal delivered through TCR and an antigen-nonspecific costimulatory signal (van Seventer et al., 1991 Curr. Opin. Immunol. 3:294; Linsley et al., 1991, J. Exp. Med. 173:721). In this regard, tumor cells may effectively evade the immune system by several mechanisms which are not only confined to tumor cells, but may also be related to impaired function of the immune response in a tumor bearing host (Gretten and Jaffee, 1999). These include: defective expression of MHC complex on tumor cells, antigen processing defects, lack of T cell recognition by outgrowth of antigen negative clones of tumor cells, inadequate expression of costimulatory molecules on tumor cells, inadequate expression of adhesion molecules on tumor cells, inadequate expression of Fas receptor and/or FaL expression on tumor cells, immune-suppressive cytokine secretion into tumor microenvironment, and host defense failure due to impaired immune cell function (Boon et al., 1997). Therefore, in the majority of cases the immune system either fails to recognize the tumor or the response that is generated is too weak to be effective. Furthermore, the management of residual and metastatic disease is a central problem in the treatment of tumors. During a normal immune response, full activation of antigen-specific naive T cells requires at least two distinct signals from surface receptors to proliferate in response to antigens (Young et al., 1992, J. Clin. Invest. 90:229; Allison and Krummel, 1995, Science 270:932). One of the signals is supplied by T cell receptor (TCR) engagement with peptide (antigen)-loaded major histocompatibility complex (MHC) molecules on antigen-presenting cells (APC). The second signal, at present poorly understood, can be delivered by the interaction of various molecules on the surface of T cells and the APC, one of which is the interaction of CD28 and B7-1 (Linsley et al, 1991; Young et al, 1992; Bluestone, 1995, Immunity 2:555). The combination of these two signals leads to activation, clonal expansion and differentiation into effector cells of T lymphocytes (Guerder et al., 1995, J. Immunol. 155:5167; Webb and Feldmann, 1995, Blood 86:3479; Thompson, 1995, Cell 81:979). Effector T lymphocytes, unlike naive T cells, no longer require costimulatory signals to recognize and kill antigen-bearing targets. After the immune response, a fraction of the effector cells remain as memory cells that form the basis of a faster and stronger immune response upon subsequent presentation of the same antigen (Gray, 1993, Ann. Rev. Immunol. 11:49; Ahmed and Gray, 1996, Science 272:54). The absence of second signal results in T cell clonal anergy, thus preventing clonal expansion of T lymphocytes (Chen, 1998; van Gool et al., 1994, Res. Immunol. 146:183).

Although many tumor cells express target antigens, they are generally incapable of stimulating an immune response (Boon et al., 1997; Boon and van der Bruggen, 1996, J. Exp. Med. 183:725). Cytotoxic T lymphocytes (CTL) have been recognized as a critical component of the immune response to tumors (Boon and van der Bruggen, 1996; Chen et al., 1994, J. Exp. Med. 179:523). CTL responses are sufficient to protect against tumors and can eliminate even established cancers in murine models (Mogi et al., 1998, Clin. Cancer Res. 4:713) and in humans (Gong et al., 2000, Proc. Natl. Acad. Sci. USA 97:2715). Inducing strong antigen-specific CTL responses is the goal of many current cancer vaccine strategies. The development of CTL-dependent anti-tumor immunization strategies depends on both the identification of tumor antigens recognized by CTLs and the development of methods for effective antigen delivery. CTL target tumors through recognition of a ligand consisting of a self MHC class I molecule and a peptide antigen generally derived from proteins synthesized within the tumor cell. However, for CTL induction and expansion to occur, the antigenic ligand must be presented to CTLs in the appropriate context of costimulation usually provided by professional APCs. Delivery of exogenous antigen to the endogenous MHC class I restricted processing pathway of professional APCs is a critical challenge in cancer vaccine design. Antigen delivery strategies currently under development include immunization with defined peptides, particulate proteins capable of accessing the class I pathway of professional APCs in vivo, heat shock proteins isolated from tumor cells, or adoptive transfer of antigen-loaded APCs. In addition, recent studies suggest that DNA vaccines encoding tumor antigens delivered by viral vectors or liposomes, or as naked DNA, can induce potent anti-tumor immunity.

In addition to the challenge of antigen delivery, most current tumor immunization strategies depend on the identification and production of appropriate tumor antigens. To overcome this limitation, tumor cells themselves may be used as immunogens as described in ACV (autologous cell vaccine). Engineering tumor cells to provide APC function could potentially result in polyvalent immunization to multiple tumor-specific epitopes, while obviating the need to identify specific tumor antigens. Many tumor vaccine strategies, including cytokine-transduced tumor cells, commonly referred to as gene therapy (Asher et al., 1991, J. Immunol. 146:3227; Tahara et al., 1996, Ann. NY Acad. Sci. 795:275; Lotze et al., 1996, Ann. NY Acad. Sci. 795:440; Rakhmilevich et al., 1997, Hum. Gene Ther. 8:1301; Nawrocki and Mackiewicz, 1998, Cancer Treat Rev. 25:29), synthetic peptide vaccine (Rosenberg et al., 1998, Nature Med. 4:321), tumor-antigen(peptide)-pulsed dendritic cells (Flamand et al, 1994, Eur. J. Immunol. 24:605; Bianchi et al., 1996, J. Immunol. 157:1589; Ashley et al., 1997, J. Exp. Med. 186:1177; Yang et al., 1997, Cell. Immunol. 179:84; Thurner et al., 1999, J. Exp. Med. 190:1669), and DNA vaccine (Leclerc and Ronco, 1998, Immunol. Today 19:300; Akbari et al., 1999, J. Exp. Med. 189:169) are currently under pre-clinical and clinical investigation but to date have yielded only marginal immunological and clinical response.

U.S. Pat. Nos. 5,635,188 and 5,993,829 teach a cancer vaccine comprising purified cell surface antigens shed by human cancer cell lines during culturing. The peptides are then used as a vaccine for immunization against cancer. That is, the antigens are used to sensitize the recipient's immune system to these antigens so that they are recognized in the event that tumors expressing these antigens develop.

U.S. Pat. No. 5,571,515 teaches the use of purified IL-12 protein in combination with an antigen from a pathogenic organism in a vaccine wherein IL-12 acts as an adjuvant to increase the vaccinated host's immune response to the pathogen. IL-12, either purified peptide or “naked” DNA encoding IL-12 or an expression vector containing IL-12 is administered with the vaccine either simultaneously or separately. This patent also describes a cancer vaccine, comprising a tumor antigen coadministered with purified IL-12.

U.S. Pat. No. 5,744,132 teaches methods for preparing formulations of IL-12 for use as a pharmaceutical. Specifically, the IL-12 protein is produced via an expression vector system and the IL-12 protein is recovered and lyophilized.

U.S. Pat. No. 5,891,680 teaches bioactive fusion proteins comprising, in one example, the p35 and p40 subunits of IL-12 in either order joined together by an intervening peptide linker. This patent mentions the fusion proteins as “potentially useful for the enhancement of anti-tumor immunity” but does not describe how this would be done.

U.S. Pat. No. 6,080,399 teaches a method wherein isolated antigen presenting cells are pulsed with a melanoma or similar peptide antigen. The isolated cells are then injected into patients as a vaccine in conjunction with IL-12 protein. The method teaches that subsequent injections with IL-12 are required for maximum efficiency of immune response induction.

As can be seen, these prior art patents teach the use of vaccines including IL-12 protein. However, in these instances, a finite amount of IL-12 is being supplied, and this supply diminishes over time as the protein is degraded. As a consequence, subsequent doses or injections of IL-12 may be required, as discussed above.

U.S. Pat. No. 5,922,685 describes DNA cancer vaccines which comprise p35 and p40 subunits of IL-12 under control of a single promoter (bicistronic transcript) as well as under the control of separate promoters. Furthermore, the inventors note that the non-bicistronic transcript vector (separate promoters for each subunit) was most effective. The inventors describe using the vaccine for noninvasive immunization, that is, to transfect epidermal cells and possibly mucosal surfaces.

As discussed above, methods requiring administration of peptides or proteins have inherent limitations, due to turn-over and degradation. Furthermore, the prior art does not teach methods for modifying tumor cells or cancer cells such that the cells express IL-12 and a costimulatory molecule so that the patient's immune system more effectively recognizes the tumor cell or cancer cell and elicits an immune response to destroy the tumor or cancer cells.

### SUMMARY OF THE INVENTION

According to a first aspect of the invention, there is provided a method of eliciting an anti-tumor immune response in a patient comprising: isolating cancerous cells from a patient; transfecting said cancerous cells with an expression vector system comprising a DNA molecule encoding IL-12 and a costimulatory molecule operably linked to a promoter capable of directing expression of said DNA molecule in said cancerous cells; incubating said transfected cells under conditions whereby the IL-12 and the costimulatory molecules are expressed; and eliciting an anti-tumor immune response in the patient by injecting said transfected cells into the patient.

According to a second aspect of the invention, there is provided a method of vaccinating an individual comprising: providing cancerous cells isolated from a donor; transfecting said cancerous cells with an expression vector system comprising a DNA molecule encoding IL-12 and a costimulatory molecule operably linked to a promoter capable of directing expression of said DNA molecule in said cancerous cells; incubating said transfected cells under conditions whereby the IL-12 and the costimulatory molecules are expressed; isolating naive T cells from the individual; exposing the T cells to the transfected cancerous cells, thereby activating the T cells; separating the active T cells from the transfected cancerous cells; and injecting the activated T cells into the patient.

According to a third aspect of the invention, there is provided an expression system comprising a DNA molecule encoding IL-12 and a costimulatory molecule operably linked to a promoter.

According to a fourth aspect of the invention, there is provided a cancerous cell transfected with the expression system described above.

### BRIEF DESCRIPTION OF THE DRAWINGS

FIG. 1 shows the overall procedure for generating DNA vector encoding IL-12 and B7-1.

FIG. 2 shows IL-12 production of IL-12 gene-transfected COS cells using ELISA.

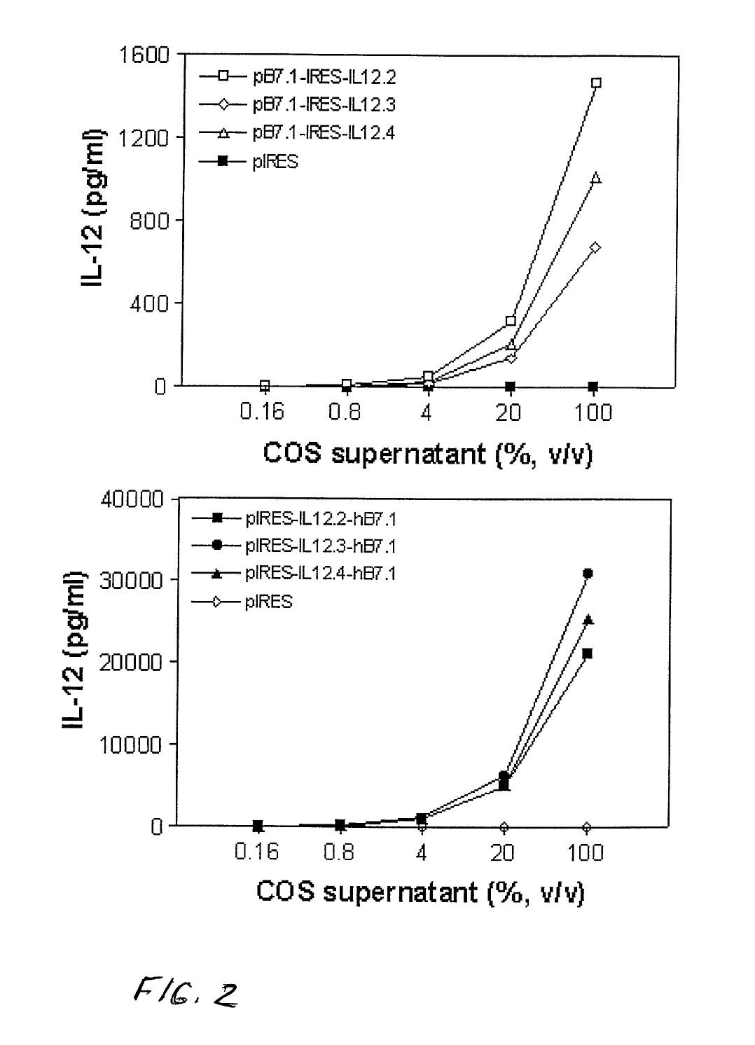

FIG. 3 shows functional analysis of IL-12 produced by gene-transfected COS cells. Augmentation of PHA-stimulated lymphocyte proliferation was compared to standard recombinant human IL-12.

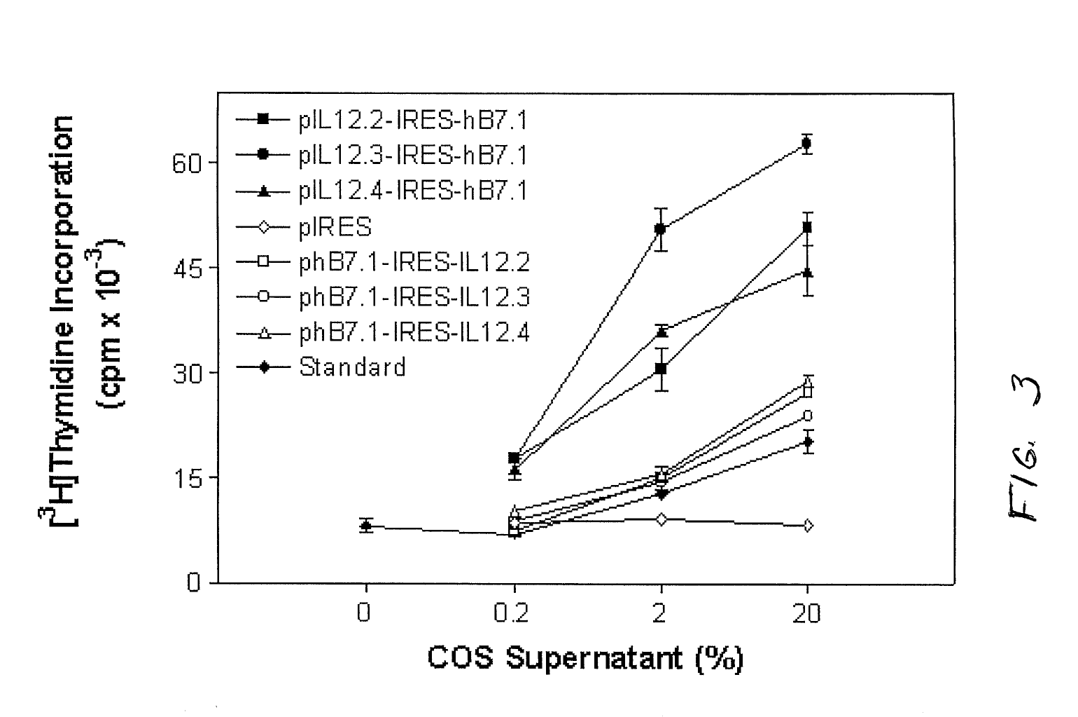

FIG. 4 shows FACS analyses of COS cell transfected with B7-1 gene. Note that unmodified or mock-transfected COS cell is negative for B7-1.

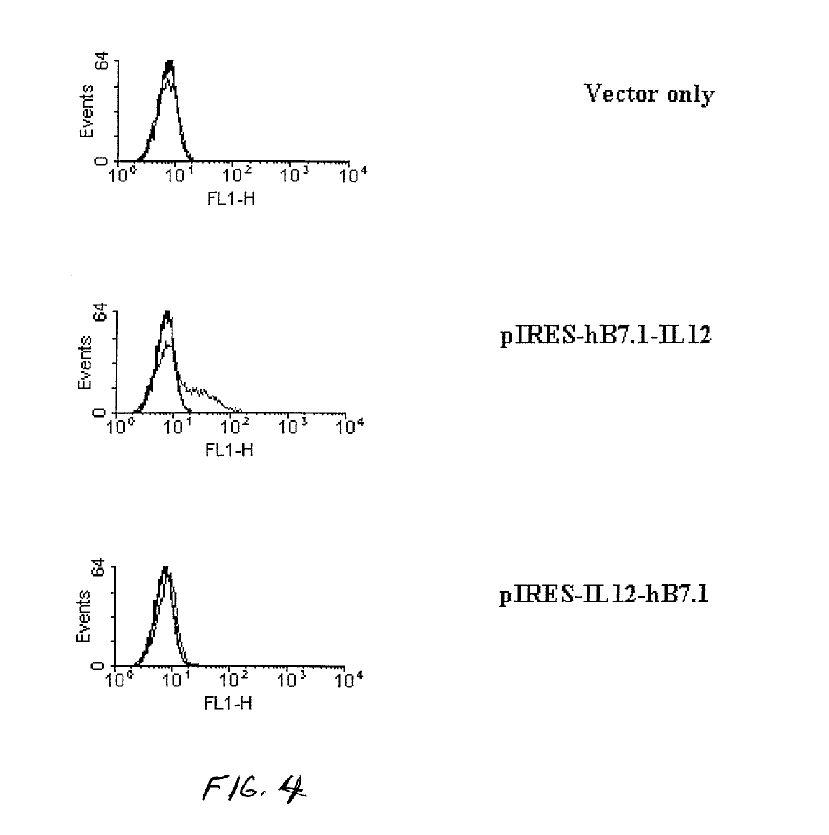

FIG. 5 shows costimulation of PHA-stimulated PBMC by B7-1 gene transfected COS cells.

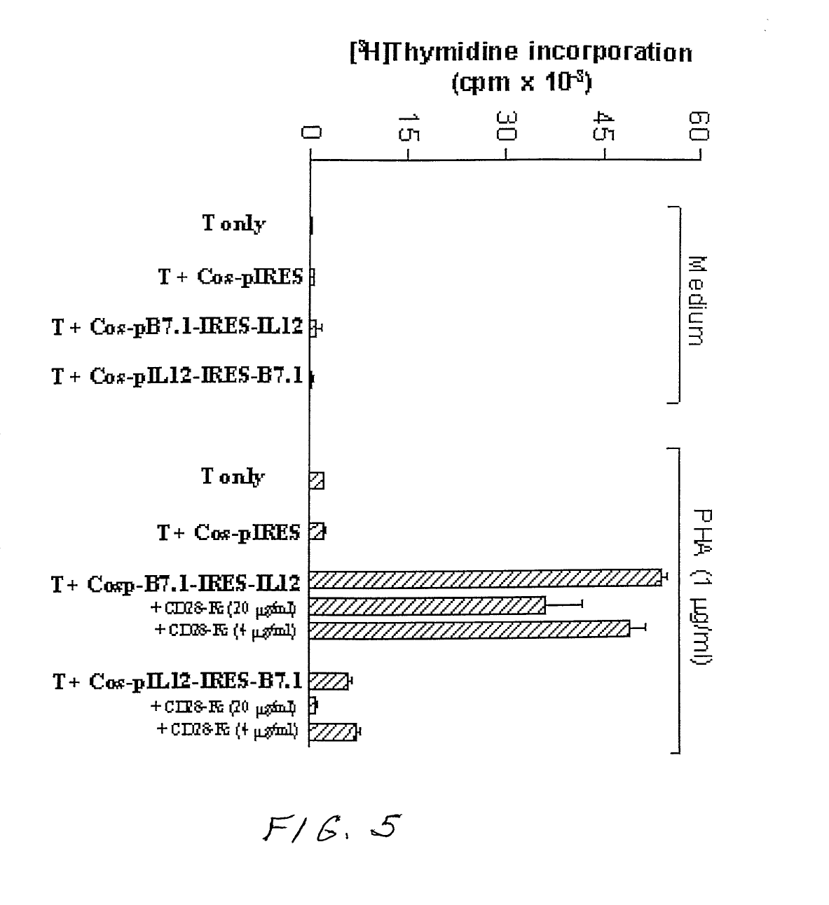

FIG. 6 shows the amino acid sequence of IL12.1 (SEQ ID No. 2).

FIG. 7 shows the amino acid sequence of IL12.0 (SEQ ID No. 4).

FIG. 8 shows the amino acid sequence of IL12.3 (SEQ ID No. 6).

FIG. 9 shows the amino acid sequence of IL12.2 (SEQ ID No. 8).

FIG. 10 shows the amino acid sequence of IL12.4 (SEQ ID No. 10).

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Definitions

As used herein, “IL-12” refers to bioactive interleukin-12. As will be appreciated by one knowledgeable in the art, this includes IL-12 assembled from p35 and p40 subunits or bioactive fragments thereof, a recombinant IL-12 comprising a fusion of p35 and p40 (or bioactive fragments thereof). The subunits may be joined by a linker.

As used herein, “costimulatory molecule” refers to molecules capable of amplifying an immune response, for example, B7-1, B7-2 and CD40L.

Described herein is a novel expression vector that comprises DNA sequences encoding IL-12 and a costimulatory molecule as well as methods of using same.

As will be appreciated by one knowledgeable in the art, any suitable control sequences (promoters, polyadenylation sites, ribosome binding sites etc) known in the art may be utilized in the expression vector.

In one embodiment, the costimulatory molecule is B7-1, although, as will be appreciated by one knowledgeable in the art, other suitable costimulatory molecules, for example, B7-2 and CD40L, may also be used.

The method of use of the vaccine comprises isolating tumor cells or cancer cells from either donors or the patient to be treated, as described below. The isolated cancer cells are transfected with the above-described expression vector and are grown under conditions such that IL-12 and the costimulatory molecule are expressed. As a result of this arrangement, the cancer cells are effectively converted to antigen presenting cells (APC). The APC cancer cells are then exposed to T cells isolated from the patient, either in vivo or ex vivo. That is, in one embodiment, the APC cancer cells are irradiated to prevent reproduction of the APC cancer cells prior to injecting the APC cancer cells into the patient. In another embodiment, T cells isolated from the patient are exposed to the APC cancer cells and are then isolated from the APC cancer cells before being injected into the patient. In both embodiments, the T cell response is activated which in turn elicits an immune response against tumors. Furthermore, once the tumor has been destroyed, memory cells remain, meaning that the patient is effectively immunized against the tumor, and a subsequent immune response will be faster and stronger.

Thus, as discussed above, the instant invention relates to the development of a new method of cancer immunotherapy and its in vitro, ex vivo, and in vivo uses. More specifically, this invention relates to the development of DNA vector comprising IL-12 and a costimulatory molecule and the protocol suitable for the in vitro generation of genetically modified human cancer cells for cancer therapy. These cells share phenotypes of both antigen presenting cells and cancer cells and are suitable as a cellular vaccine for certain types of cancer.

Since most tumor cells do not express costimulatory molecules, tumor-specific antigens are not presented to T cells efficiently (Denfeld et al., 1995; Gajewski et al., 1996, J Immunol. 156:2909). Indeed, this may represent one mechanism by which tumor cells elude recognition by the immune system. Therefore, costimulatory molecules have been expressed on the surface of tumor cells to enable them to present tumor-associated antigens, together with the costimulatory signal, directly to T cells thus obviating the need for helper T cells and APCs. In support of this model, at least in primary responses against tumors in vivo, some B7-1-expressing tumors have been found to elicit an effective response that is mediated by CD⁺ and CD⁺ T cells (Dranoff et al., 1993, Proc. Natl. Acad. Sci. USA 90:3539). In contrast, nonimmungenic tumors failed to lose tumorigenicity with the transduction of B7-1 alone (Chen et al., 1994). On the basis of these experiments, we can speculate that vaccination of tumor patients with B7 autologous tumor cells will only boost antitumor responses when the tumors are immunogenic, i.e., expresses sufficient amounts of tumor peptide-loaded MHC class-I or class-II molecules that can be recognized by the patients' T cells. An additional costimulatory signal seems to be required for inducing antitumor effector cells against poorly immunogenic tumors. In this regard, augmentation of antitumor immune responses might then be obtained by co-expressing B7 with a cytokine which up-regulate MHC molecules, attract and stimulate other immune effector cells. IL-12 has been selected for its pleiotropic effects on a variety of immune cell types and also because B7-1 and IL-12 co-operate in stimulating lymphocyte proliferation and activation in vitro (Trinchieri, 1998, Adv. Immunol. 70:83). This cytokine induces a strong local response against a number of tumors, in the context of using recombinant IL-12 paracrine secretion at the tumor site using engineered fibroblasts (Zitvogel et al., 1995, J. Immunol. 155:1393) or transformed tumor cells (Tahara et al, 1995, J. Immunol. 154:6466), as well as direct in vivo gene delivery (Rakhmilevich et al., 1997). IL-12 also elicits protective immunity against poorly immunogenic tumors and was effective in causing regression of pre-established tumors (Tahara, et al., 1995, J. Immunol. 154:6466; Cavallo et al., 1997, J. Natl. Cancer lnst. 89:1049). IL-12 stimulates the proliferation of activated T and NK cells, synergizes with IL-1 in the generation of lymphokine-activated killer cells (LAK) and enhances their lytic activity (Gately et al., 1991, J. Immunol. 147:874). Its ability to stimulate directly the production of IFN-γ both in vitro (Chan et al., 1991, J. Exp. Med. 173:869) and in vivo (Nastala et al., 1994, J. Immunol. 153:1697) and to induce primarily a Th1 response in vitro suggests its potential utility as an antitumor agent. Indeed, recent studies (Tahara et al., 1996; Brunda et al., 1996, Ann. NY Acad. Sci. 795:266) implementing systemic administration of the rIL-12 protein had profound effects on virtually every murine tumor model evaluated. Systemic administration of murine rIL-12 in several tumor models mediates profound T cell-mediated antitumor effects in vivo, leading to regression of established tumor masses, which is frequently associated with the generation of antitumor immunological memory. By the same token, however, prolonged systemic administration of rIL-12 has been related with systemic toxicity (Motzer et al., 1998, Clin. Cancer Res. 4:1183.). To address this problem, several studies on experimental tumors have evaluated the therapeutic potential of IL-12-based tumor cell vaccines and, with few exceptions, the vaccines were found to cure or significantly improve the survival of mice bearing a variety of tumors. The antitumor activity of systemic or local release of IL-12 is largely mediated by IFN-γ secreted at the tumor site by stimulated NK cells ad T cells, along with up-regulation of MHC expression on tumor cells, NOS induction, release of other cytokines, and inhibition of angiogenesis through the induction of the chemokine IP-10 by both tumor cells and infiltrating immune cells (Trinchieri, 1998).

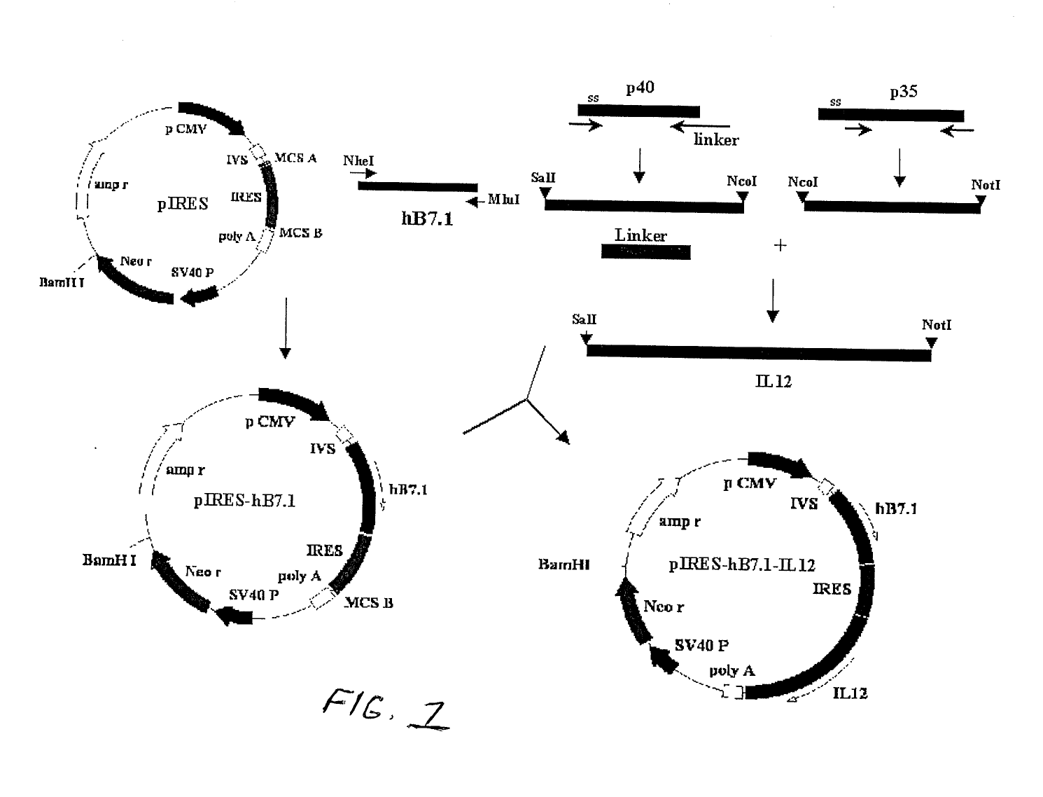

The heterodimeric structure of IL-12 substantially complicated the construction of vectors for IL-12 production, because the expression of bioactive IL-12 requires the expression of two separate genes and subsequent correct heterodimeric assembly of the subunits (Mattner et al., 1993, Eur. J. Immunol. 23:2202). In vivo, activity of IL-12 requires the expression of the p35 and p40 subunits, which are located on different chromosomes which are regulated independently (Wolf et al., 1991, J. Immunol. 146:3074). Furthermore, it has been shown that excessive p40 subunit specifically antagonizes the effects of the IL-12 heterodimer in vitro (Mattner et al, 1993). We therefore designed a single chain recombinant IL-12 molecules by genetic recombination. Specifically, in order to achieve balanced expression of both subunits of IL-12 (p35 and p40), we generated a single chain p70 molecule by joining both subunits with various size of flexible linker. The Gly-Gly-Gly-Gly-Ser repeats were chosen for their flexibility and also because it had been used previously in constructing single chain antibodies and PIXY-321 (Curtis et al., 1991, Proc. Natl. Acad. Sci. USA 88:5809), a fused form of GM-CSF and IL-3 already in clinical use. As shown in FIG. 1, IL-12 and B7-1 were inserted into a bicistronic vector which contains an internal ribosome entry site (IRES).

In the embodiment shown in FIG. 1, the costimulatory molecule is B7-1, which enhances sensitization and activation of tumor-reactive CD⁺ T cells (Guerder et al., 1995); however, costimulatory molecules are not required for recognition and destruction of tumor cells by activated effector T cells (CTL). Moreover, it is clear that IL-12, even when present at pg/ml concentration, has profound effects on the generation of human CTLs in synergy with B7-1 stimuli (Komata et al., 1997, J. Immunother. 20:256). That is, direct activation of CTLs by tumor cells expressing B7-1 and IL-12 induces antitumor responses, bypassing the need for exogenous CD4+T cells or APCs or both, which leads to a faster and more effective antitumor response in situ. This may provide a means of eliciting effective CTL responses to tumors in patients. Thus, immunotherapy of the tumor aims to generate an effective systemic immune response capable of controlling the growth of metastatic tumors.

Our vaccine, ACV (autologous cell vaccine), utilizes cell lines derived from tumors that are modified and administered in ways that substantially enhance the response by the immune system. The cells used in the manufacture of the vaccine are sterilized by irradiation prior to administration to ensure that they are unable to replicate. The costimulatory molecule signals the immune cells in the treated patients while IL-12 recruits immune cells to the tumor site(s), and kills tumor cells by preventing angiogenesis. Our vaccine therapy would supplement existing modalities such as chemotherapy, radiotherapy and surgery by using the immune system to contain or destroy residual tumor cells, thereby increasing the length of remission, or preventing recurrence of the cancer. The immune response will be selective for the tumor and will be capable of hunting down cancer cells throughout the body.

This approach has several advantages over other above mentioned approaches that (a) it does not require the identification and purification of antigenic peptides, (b) it can be applied to almost every types of cancers, and (c) it may not induce GvHD since cancer cells are derived from self. Thus, this method will provide a new and safe therapeutic strategy for primary and metastatic cancer by activating patient's own immune system.

The present invention provides methods and compositions for use of genetically modified cancer cells to activate T cells for immunotherapeutic responses against primary or metastatic cancer. The cancer cells obtained from human donors, after transfection or transduction with the IL-12 and B7-1 expression vector described above, are administered to a cancer patient to activate the relevant T cell responses in vivo. Alternatively, T cells from patients are exposed to genetically modified cancer cells in vitro to activate the relevant T cell responses in vitro. The activated T cells are then administered to a cancer patient. In either case, the genetically modified cancer cells are advantageously used to elicit an immunotherapeutic growth-inhibiting response against a primary or metastatic tumor.

Examples of suitable vectors are discussed below and are shown in SEQ ID Nos. 1, 3, 5, 7, 9, 11, 13 and 15. As will be apparent to one knowledgeable in the art, other suitable vectors may also be used.

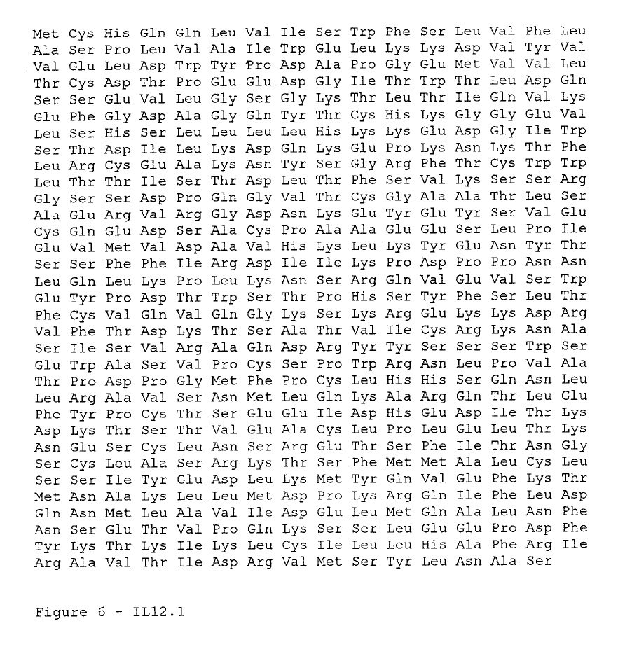

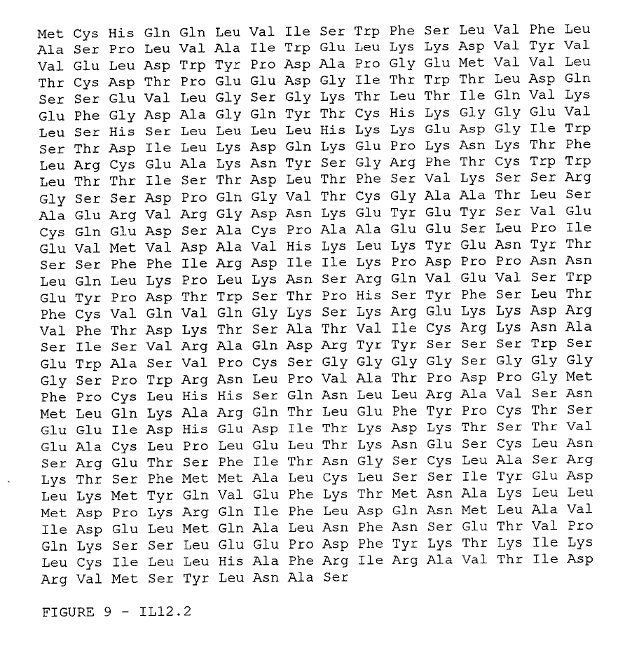

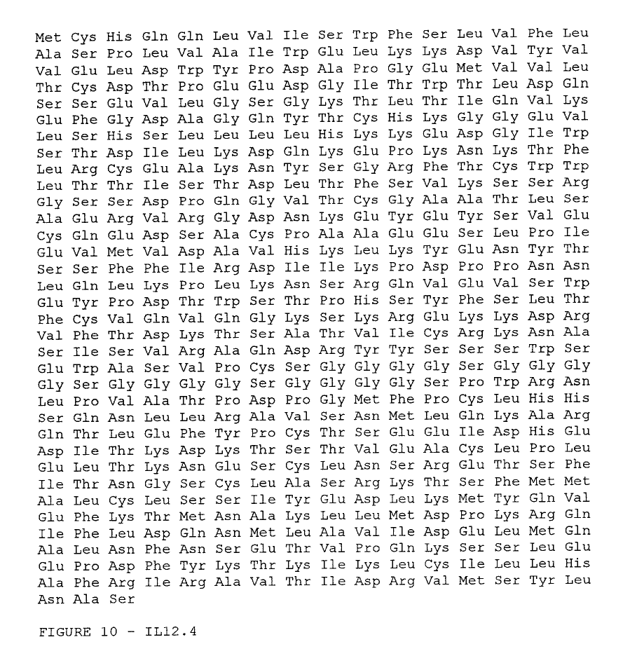

IL-12 fusions are shown in FIGS. 6-10 and in SEQ ID Nos. 2, 4, 6, 8, 10, 12, 14 and 16 and are discussed below.

### EXAMPLE I

Construction of Vectors Encoding hB7-1 and Single Chain IL-12

Human IL-12 gene and B7-1 genes are obtained from antigen presenting cells of various tissues, for example, the spleen, bone marrow and lymph nodes as well as the circulatory system including blood and lymph. It is of note that human peripheral blood is an easily accessible ready source of antigen presenting cells and is used as a source according to a preferred embodiment of the invention. Cord blood is another source of human antigen presenting cells.

Because antigen presenting cells that express both IL-12 and B7-1 spontaneously exist in low numbers in any tissues in which they reside, including human peripheral blood, antigen presenting cells must be enriched or isolated for use. Any of a number of procedures, for example, repetitive density gradient separation, positive selection, negative selection or a combination thereof may be used to obtain enriched populations or isolated antigen presenting cells.

Once the antigen presenting cells are obtained, they are cultured in appropriate culture medium to stimulate the expression of IL-12 and B7-1. Particularly advantageous for inducing the proper state of antigen presenting cells in in vitro culture is the presence of LPS for monocytes, Anti-Ig and IL-4 or CD40 ligand (CD154) and IL-4 for B cells, or GM-CSF, IL-4 and CD40L for dendritic cells Preferred cells and conditions are of monocytes and LPS at concentration of 100-1000 ng/ml.

According to a preferred embodiment, both B7-1 and IL-12 genes have been cloned by standard recombinant DNA techniques. The cDNA encoding the p35 and p40 chains of human IL-12 and human B7-1 were generated from LPS-stimulated human peripheral blood monocytes by reverse transcriptase-PCR.

Primers selected from the 5′ and 3′-end of the coding sequences of each gene were designed to introduces the appropriate restriction sites for further genetic recombinations, as shown in FIG. 1.

After cloning of B7.1 and IL-12, each gene was subcloned into a vector.

Specifically, B7-1 was excised at appropriate restriction sites and inserted into MCS A site on pIRES excised with corresponding restriction digests or into MCS B site excised with corresponding restriction enzyme generating pIRES-hB7-1 (A) and pIRES-hB7-1(B).

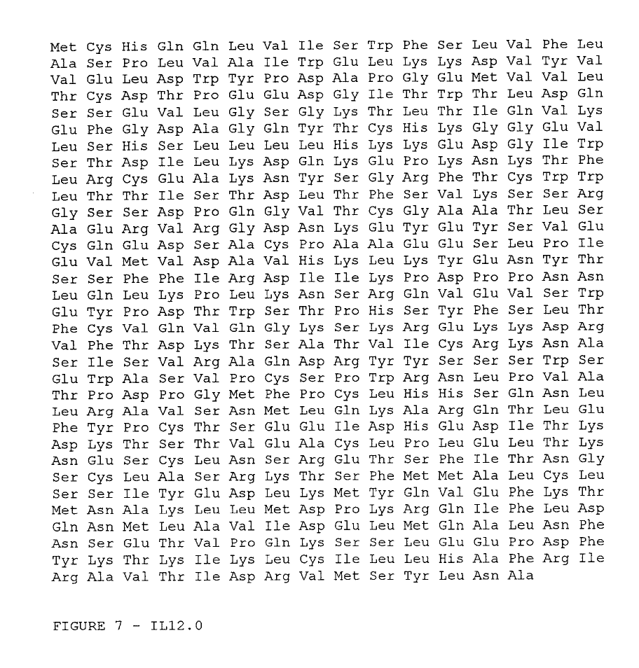

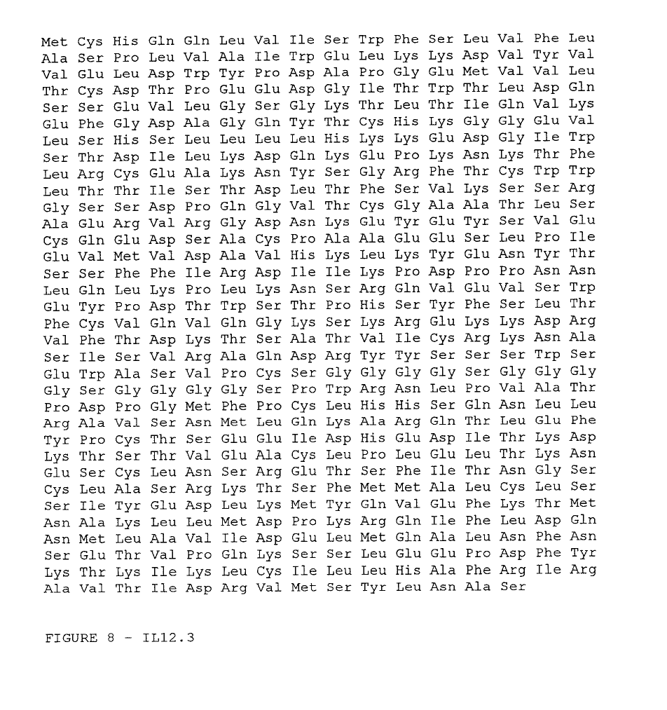

The cDNA for the single chain IL-12 fusion protein was constructed by linkage of the p40 and p35 cDNAs with a synthetic flexible linker. We have utilized three different linkers that have 2, 3 and 4 repeats of Gly-Gly-Gly-Gly-Ser (shown in FIGS. 9, 8 and 10, respectively). These products (about 1.6 kb) have been cloned into pGEM-T easy vector generating pGEM-IL12.2, pGEM-IL12.3, and pGEM-IL12.4. PCR products from these three vectors were digested with appropriate sets of restriction enzyme and ligated into the MCS B on pIRES-hB7-1 (A) or into the MCS A on pIRES-hB7-1(B) generating 2 series (pIRES-hB7-1-IL12 and pIRES-IL12-hB7-1 series) of 6 different expression vectors (pIRES-hB7-1-IL12.2 (SEQ ID No. 7), pIRES-hB7-1-IL12.3 (SEQ ID No. 5), and pIRES-hB7-1-IL12.4 (SEQ ID No. 9); pIRES-IL12.2-hB7-1 (SEQ ID No. 15), pIRES-IL12.3-hB7-1 (SEQ ID No. 13), and pIRES-IL12.4-hB7-1 (SEQ ID No. 11)). In addition, constructs with a single linker (IL12.1, shown in FIG. 6; SEQ ID No. 2) and no linker (IL12.0, shown in FIG. 7; SEQ ID No. 4) were used to construct pIRES-hB7.1-IL12.1 (SEQ ID No. 1) and pIRES-hB7.1-IL12.0 (SEQ ID No. 3) respectively. Constructs were sequenced across all cloning junctions to determine the fidelity of recombination process.

### EXAMPLE II

Gene Transfer into Cancer Cells

According to the present invention, the pIRES DNA vectors are introduced into a desired cancer cells by lipofectamin. After gene transfection, cells are analyzed to assess the expression of both IL-12 by ELISA and B7-1 by FACS.

Alternatively, DNA vectors can be introduced into cancer cells by other means known in the art, for example, PEG, electroporation, DEAE-dextran method or calcium phosphate method.

After 24-72 hr culture, supernatants were collected for IL-12 ELISA and bioassay and cells were collected for B7-1 expression and costimulation assay.

### EXAMPLE III

Applications or Methods of use of Genetically Modified Cancer Cell's to Stimulate T Cells Against Cancer in vitro and in vivo

According to a preferred embodiment of the invention, cancer tissues are obtained from a patient to be treated to generate a cancer cell line. The cell lines are in turn used to activate autologous T cells of the patient, either in vitro or in vivo, for cancer immunotherapy and/or tumor growth inhibition.

According to another embodiment, a tumor cell recovered from surgical specimens without further treatment can be used for gene transfer, as discussed herein.

Using an approach wherein the patient's own cancer cells that are genetically modified provides the following advantages: (A) identification of cancer antigen is not required; (B) temporal expression of genes eliminates dangerous side effects; (C) antigen on cancer cells along with costimulatory factor, B7-1, is presented to T lymphocytes; (D) the use of B7-1 on and IL-12 from cancer cell surface eliminates the need to provide T cells with IL-2 or other cytokines either in the form of the cytokine itself or transfection of the cDNA into specific cells; (E) all procedures are carried out using the patient's own cells.

### EXAMPLE IV

Cell Lines and Reagents

Cell lines are established from pathologically proven cancer tissues. Soild tumors were first finely minced with scissors and dissociated into small aggregates by pipetting. Appropriate amounts of fine neoplastic-tissue fragments were seeded into 25 cm² flasks. Tumor cells were initial cultured in RPMI-1640 medium containing 10% heat-inactivated fetal calf serum. After establishment, passages were performed when heavy tumor cell growth is observed. If stromal cell growth is noted in initial cultures, differential trypsinization is used to obtain a pure tumor cell population. Established human cancer cells are maintained in culture in RPMI 1640. Mouse monoclonal anti-human CD80 and isotype-matched control antibodies were purchased from Becton-Dickinson, San Jose, Calif.

### EXAMPLE V

Cancer Patients

Patients with a histologic confirmation of cancer are selected for this study which included a signed informed consent. 50 ml of heparinized peripheral blood were drawn every 2 weeks during the period of observation which continues. Details regarding clinical stage, hematologic status, and other relevant treatments are recorded.

### EXAMPLE VI

Isolation of Monocytes and Stimulation With LPS

Peripheral blood was drawn from normal donors and was subjected to Ficoll-Hypaque (Pharmacia, Uppssala, Sweden) density gradient centrifugation. After washing twice with Hank's Balanced salt solution (HBSS, Life Technologies, Grand Island, N.Y.), monocytes were separated according to their plastic adherence (Steinbach et al., 1998, Res. Immunol. 149:627; Thurner et al., 1999, J. Immunol. Methods 223:1) for 30 min -1 hr incubation in 5% CO₂, 37° C. incubator. Enriched monocytes were cultured in the presence of 100-1000 ng/ml of LPS (Sigma Chem. Co., St. Louis, Mo.). Alternatively, monocytes are purified from eripheral blood mononuclear cells by positive selection by CD14-MACS column (Miltenyi Biotec). After 16-24 hr of incubation, cells were treated with Trizol (Life Technologies) for RNA preparation and subsequent gene cloning.

### EXAMPLE VII

Construction of Vectors Encoding HB7-1 and Single Chain IL-12

Both B7-1 and IL-12 genes were cloned by standard recombinant DNA techniques, as discussed herein. The cDNA encoding the p35 and p40 chains of human IL-12 and human B7-1 were generated from 1 μg/ml of LPS-stimulated human peripheral blood monocytes or CD40 ligand and IL4 stimulated human B cells by reverse transcriptase- PCR. Typically, peripheral blood monocytes were utilized for experimental convenience. First, mRNA was isolated by oligo-dT MACS column (Miltenyi Biotec) and subjected to first strand cDNA synthesis. Primers selected from the 5′ and 3′-end of the coding sequences of each gene were designed to introduces restriction sites. PCR was done with PCR-premix (Bioneer, Cheongwon, Chungbuk, Korea) containing PCR buffer, dNTP, Taq polymerase and MgCl₂. After cloning of B7.1, each gene has been subcloned into PGEM vector generating pGEM-hp40, pGEM-hp35, and pGEM-hB7-1 and clones were verified by sequencing.

Next, the PCR product of hB7-1 was either excised at appropriate restriction sites and inserted into MCS A site on pIRES or into MCS B site excised with restriction enzymes generating pIRES-hB7-1 (A) and pIRES-hB7-1(B).

The CDNA for the single chain IL-12 fusion protein was constructed by linkage of the p40 and p35 cDNAs with synthetic linkers of 2, 3 and 4 repeats of Gly-Gly-Gly-Gly-Ser containing NcoI restriction sites. The p40 from pGEM-hp40 has been amplified with linker-containing primers producing p40L2, p40L3, and p40L4. The p35 was cloned downstream of the linker with restriction site. These products (about 1.6 kb) have been cloned into pGEM-T easy vector generating pGEM-IL12.2, pGEM-IL12.3, and pGEM-IL12.4. PCR products from these three vectors were digested with the appropriate restriction enzymes and ligated into the MCS B on pIRES-hB7-1 (A) or into restriction site of MCS A on pIRES-hB7-1(B) generating 2 series (pIRES-hB7-1-IL12 and pIRES-IL12-hB7-1 series) of 6 different expression vectors (pIRES-hB7-1-IL12.2, pIRES-hB7-1-IL12.3, and pIRES-hB7-1-IL12.4; pIRES-IL12.2-hB7-1, pIRES-IL12.3-hB7-1, and pIRES-IL12.4-hB7-1). Constructs were sequenced across all cloning junctions to determine the fidelity of recombination process.

### EXAMPLE VII

Transfection of Plasmid Encoding IL-12/B7-1 Genes and Analysis of Gene Expression

COS-7 cells or human cancer cell lines in 6-well plates at 60-70% confluency were transfected with 5 μg of vector prepared by Qiagen plasmid kit (Qiagen Inc, Valencia, Calif.) using Lipofectin (Life Technologies) according to manufacturer's instructions. After 5 hr incubation at 37° C., the DNA/Lipofectin mixture was removed and replaced with fresh medium. After 48 hr, supernatants were collected for IL-12 ELISA and bioassay, and cells were collected for B7-1 expression. IL-12 in the culture supernatants of transfected cells was quantitated using human IL-12 p70 ELISA kit (Endogen, Woburn, Mass.). The function of IL-12 produced by gene transfected cells can be analyzed by PHA-blast assay (Gately et al., 1991) and/or IFN-γ induction assay of PHA-stimulated PBMC. B7-1 expression on gene-transfected cells can be analyzed by flow cytometer after staining with monoclonal anti-B7-1 antibody, while the function of B7-1 can be analyzed by proliferation of PBMC stimulated with sub-optimal dose of PHA in the presence of gene-transfected cells.

### EXAMPLE IX

Collection of Tissue and Preparation Cells

Patients with a histologic confirmation of cancer were selected for this study which included a signed informed consent. 50 ml of heparinized peripheral blood was drawn. Details regarding clinical stage, hematologic status, and other relevant treatments were recorded. Cancer cell lines were established from patients. Solid tumors were finely minced with scissors and dissociated into small aggregates by pipetting. Appropriate amounts of fine tissue fragments were seeded into 25 cm² flasks. In most cases, tumor cells were cultured in RPMI-1640 medium supplemented with 10% heat inactivated FCS. Initial passages were performed when heavy tumor cell growth was observed; subsequent passages were performed once or twice a week. Adherent cell cultures were passaged at sub-confluency after trypsin-EDTA (Life Technologies) treatment, and floating cells were passaged after dissociation, if necessary, of the cells by pipetting. If stromal cell or fibroblast growth was noted in initial cultures, differential trypsinization was used to obtain a pure tumor cell population. All cultures were maintained in humidified incubators at 37° C. in an atmosphere of 5% CO₂ and 95% air. Phenotypes of established cell lines were analyzed with FACS (Becton Dickinson) after fluorochrome-labeled monoclonal antibody staining.

### EXAMPLE X

Construction of DNA Vectors Encoding hB7-1 and Single Chain IL-12

Mononuclear cells were isolated by Ficoll-Paque (Pharmacia) density gradient centrifugation of heparinized blood obtained from healthy adult donors according to standard protocols. Then, cells (5×10⁶/ml, 10 ml) resuspended in RPMI-1640 medium supplemented with 10% FCS, 10 mM of glutamine, and penicillin/streptomycin and subjected to 1 hr plastic adherence. Non-adherent mononuclear cells were removed by rinsing with warm PBS and remaining monocytes were stimulated with LPS(1 μg/ml, Sigma) for 20 hr at 37° C., 5% CO₂ incubator. Both B7-1 and IL-12 genes have been cloned by standard recombinant DNA techniques. The cDNA encoding the p35 and p40 chains of human IL-12 and human B7-1 were generated from LPS-stimulated human peripheral blood monocytes by reverse transcriptase-PCR. Primers selected from the 5′ and 3′-end of the coding sequences of each gene were designed to introduces SalI, NcoI, NotI, NheI and MluI restriction sites. After cloning of B7.1, each gene has been subcloned into pGEM vector generating pGEM-hp40, pGEM-hp35, and pGEM-hB7-1 and clones were verified by sequencing.

The sets of 5′ and 3′ primers for PCR were hp4F 5′-CTAGCTAGCGGCCCAGAG CMGATGTG-3′, hp4° F.b 5′-ACGCGTCGACGGCCCGAGCMGATGTG-3′, and hp40R1 5′-ACTGCAGGGCACAGATGC-3′ for p40, hp35F 5′-CATGCCATGGAG M ACCTCCCCGTGGC-3′, hp35R 5′-CCGACGCGTACCTCGCTTTTTAGGAAGCAT-3′, and hp35Rb 5′-ATMGAATGCGGCCGCACCTCGCTTTTTAGGAAGCAT-3′ for p35, and hB7-1 F 5′-ACGAGTCGACATGGGCCACACACGGA-3′, hB7-1 R 5′-GGCGGCC GTTTCAGCCCCTTGCTTTT-3′, hB7. 1 Fb 5′-CTAGCTAGCATGGGCCACACACGG A-3′, and hB7.1 Rb 5′-CCGACGCGTTTTCAGCCCCTTGCTTCT-3′ for B7.1

Next, PCR product of hB7-1 has been either excised at NheI/MluI or at Sall/Notl and inserted into MCS A site on pIRES excised with NheI/MluI or into MCS B site excised with SalI/NotI generating pIRES-hB7-1 (A) and pIRES-hB7-1(B).

The cDNA for the single chain IL-12 fusion protein was constructed by linkage of the p40 and p35 cDNAs with a synthetic linker containing Ncol restriction sites. Previous studies suggested that an accessible N-terminus of the p40 subunit is important for IL-12 bioactivity. When the p35 subunit came before the p40 subunit, there was greatly decreased IL-12 activity, in contrast, when the subunits were reversed, with p40 in front of p35, the single chain IL-12 had biological activity comparable to rIL-12.

We have utilized three different linkers that have 2, 3 and 4 repeats of Gly-Gly-Gly-Gly-Ser. The p40 from pGEM-hp40 has been amplified with linker-containing primers producing p40L2, p40L3, and p40L4. The p35 was cloned down stream of the linker with Ncol restriction site. These products (about 1.6 kb) have been cloned into pGEM-T easy vector generating pGEM-IL12.2, pGEM-IL12.3, and pGEM-IL12.4. PCR products from these three vectors were digested with SalI/NotI or NheI/MluI and ligated into the MCS B on pIRES-hB7-1(A) or into NheI/MluI site of MCS A on pIRES-hB7-1(B) generating 2 series (pIRES-hB7-1-IL12 and pIRES-IL12-hB7-1 series series) of 6 different expression vectors (pIRES-hB7-1-IL12.2, pIRES-hB7-1-IL12.3, and pIRES-hB7-1-IL12.4; pIRES-IL12.2-hB7-1, pIRES-IL12.3-hB7-1, and pIRES-IL12.4-hB7-1). Constructs were sequenced across all cloning junctions to determine the fidelity of recombination process.

In some cases, peripheral blood monocytes were subjected to MACS purification to further enrich monocyte fraction. Cells stained with CD14-microbeads (Milteyi Biotec) in the presence of FcR blocking reagent (human IgG) for 30 minutes at 4° C. After washing cells applied to MS+ column which was subjected to magnetic fields. Cells remaining in the column were collected and washed once before undergoing culturing as indicated above.

### EXAMPLE XI

IL-12 Production by Gene-Modified COS Cells

IL-12 in the culture supernatants of transfected cells was quantitated using human IL-12 p70 ELISA kit (Endogen). After transfection, culture supernatants of COS cells were harvested, filtered with 0.22 μm syringe filter, and stored until analyzing the quantity and quality of gene expression.

While cells transfected with pIRES-hB7-1-IL12 series(pIRES-hB7-1-IL12.2, pIRES-hB7-1-IL12.3, and pIRES-hB7-1-IL12.4) produced 0.6-1.5 ng/ml, culture supernatants from cells with pIRES-IL12-hB7-1 series (pIRES-IL12.2-hB7-1, pIRES-IL12.3-hB7-1, and pIRES-IL12.4-hB7-1) contained 21-32 ng/ml of IL-12 according to ELISA analysis.

All three vectors with different length of flexible linkers produced similar quantities of IL-12 indicating that the linker length did not influence the level of gene expression or conformation of IL-12.

### EXAMPLE XII

Bioactivity Measurement of IL-12 Produced by Transfected COS Cells

Assays for IL-12 induced proliferation of PBMC were performed as previously described (Gately et al., 1991, J. Immunol. 147:874.). PBMC were isolated from normal blood donors by Ficoll-Hypaque density centrifugation (Pharmacia, Piscataway, N.J.). These cells were then depleted of monocytes by plastic adherence, and non adherent cells were resuspended at 5×10⁵ cells/ml in RPMI-1640 supplemented with 10% FCS, penicillin, streptomycin, L-glutamine containing 2 μg/ml of PHA(Sigma) and were cultured for 3 days. Cells were then washed and recultured with rIL-2 (20 iu/ml) for an additional 48 hr. The PHA-blasts were then washed with acidified RPMI-1640 (pH 6.4) and rest for 4 hr in RPMI-1640 supplemented with 0.5% human AB serum. The cell concentration was adjusted to 2×10⁶ cells/ml in complete medium. 50 μl of serial 1:5 dilutions of IL-12 standard (Leinco Tech) and culture supernatants of transfected cells were made in complete medium over a range of 0.16 pg to 200 pg/mi. Next, 50 μl cell suspension (PHA-blasts) was mixed with these serial diutions in triplicate in a flat-bottomed 96-well tissue culture plate. Neutralizing anti-IL-2 antibody was included to block IL-2-induced proliferation. [³H]-Thymidine (1 μCi/well, NEN, Boston, Mass.) was added to the wells after 36 hr of culture at 37 C, 5%CO₂. After an additional 16 hr of incubation, cultures were harvested onto glass filters, and radioactivity was assessed by liquid scintillation.

The bioactivity of IL-12 produced by vector-transfected cell lines was also measured using an assay based on the ability of this cytokine to stimulate production of IFN-γ by lymphocytes. PBMC activated with PHA 5 ug/ml for 5 days were collected, washed and restimulated with serial dilutions of standard IL-12 (1-1000 pg/ml) and culture supernatants from transfected cell lines stimulated. Following a 20 hr incubation, culture supernatants of PBMC were harvested and assayed for IFN-γ by ELISA (Endogen). The assay for IL-12 was quantified by comparing the amount of IFN-γ produced in the test samples with that induced by the recombinant IL-12 standards.

In accordance with ELISA data, the bioactivity of IL-12 in the culture supernatants of pIRES-hB7-1-IL12 series was significantly lower than that of pIRES-IL12-hB7-1 series. In PHA-blast assays, IL-12 produced by transfected COS cells efficiently induced the proliferation of T cells and NK cells comparable to the standard IL-12. Moreover, in IFN-γ induction assay, the specific activity of IL-12 produced by vector transfected cells at equal concentration was almost identical or even better to rIL-12 standard.

### EXAMPLE XII

B7-1 Expression on Gene-Modified Cells by Flow Cytometric Analysis

Assessment of B7-1 on vector transfected COS-7 was done with the aid of FITC-conjugated anti-B7-1 (Pharmingen, San Diego, Calif.). As a control, we used an appropriate FITC-conjugated isotype-matched normal IgG1 (Pharmingen). Flow cytometric analysis of 10,000 viable cells was conducted on a FACS vantage (Becton Dickinson). Each experiments was repeated at least three times, and the results of a representative experiment are provided in the form of a histogram.

While a significant proportion (20-30%) of cells transfected with pIRES-B7-1-IL12 series expressed B7-1, little (less than 5%) was shown on cells transfected with pIRES-IL12-hB7-1 series.

### EXAMPLE XIII

B7-1-induced Costimulation

T lymphocytes were purified with T cell enrichment kit (Miltenyi Biotech). Allogneic T lymphocytes (1×10⁶ cells/ml) were co-cultured with irradiated (3000 rad) vector-transfected ME-180 (1, 2, 4, 8×10⁴ cells/ml) in 96 well flat-bottomed plates in the presence of 1 ug/ml of PHA. In some cases, murine CD28-Fc (Chemicon) was included to determine the direct effects of B7-1 expressed on vector-transfected ME-180. 48 hr later, each well received 1 μCi [³H]-thymidine, and after an additional 16 hr, cultures were harvested onto glass filters, and radioactivity was assessed by liquid scintillation.

While a significant T cell costimulatory activity (50-100 fold higher cpm over background) was shown by cells transfected with pIRES-B7-1-IL12 series, only a minor (3 fold higher cpm over background) was detected on cells transfected with pIRES-IL12-hB7-1 series. Furthermore, there was a dose-dependent inhibition of costimulatory activity by chimeric CD28-Fc protein, suggesting the major costimulation was provided directly by B7-1 on vector-transfected ME-180.

### EXAMPLE XIV

Cytotoxicity Assay

PBMC (1×10⁷ cells) from normal donors were stimulated with ME-180 in a volume of 10 ml. After 6 days, effector cells were collected, adjusted to 2×10⁶ cells/ml, and titrated in triplicate in flat-bottomed plates to give the indicated E:T ratios along with vector-transfected ME-180 cells. Supernatants were collected after 4 hr and cytotoxicity was measured by the kit purchased from Roche (Manheim, Germany). Percentage-specific lysis was calculated according to the manufacturer's instructions.

While a significant cytotoxicity was shown by PBMC co-cultured with ME-180 cells transfected with pIRES-B7-1, only a minor cytotoxicity was detected on PBMC co-cultured with ME-180. The co-toxic effects of PBMC co-cultured with ME-180 cells transfected with pIRES-B7-1 were exerted not only to pIRES-B7-1-IL12 series and pIRES-IL12-B7-1 series but also to unmodified ME-180, suggesting the maturation of effector CTL by B7-¹⁺ ME-180 during co-culture and implicating the immunotherapeutic potential of ACV.

Once generated, IL-12 and B7-1 genetically-modified cancer cells can, if desired, be injected by any standard method into the patient. Preferably, the cells are injected back into the same patient from whom the source cancer cells were obtained. The injection site may be subcutaneous, intraperitoneal, intramuscular, intradermal, or intravenous. The number of cells injected into the patient in need of treatment is according to standard protocols, e.g., 1×10⁴ to 1×10⁶ are injected back into the individual. The number of cells used for treating any of the disorders described herein varies depending upon the manner of administration, the age and the body weight of the subject, and the condition of the subject to be treated, and ultimately will be decided by the attending physician.

The methods and media compositions described herein are useful for the culture and production of cancer cells and gene-modified cancer cells in vitro and in vivo. Given the capacity of gene-modified cancer cells to elicit strong antigen-specific helper and cytotoxic T cell responses, the ability to generate large numbers of homogeneous preparations of cancer cells facilitates the manipulation of these cells for the development of a variety of therapies, including without limitation, ex vivo and in vivo human therapies.

The invention therefore encompasses the use of gene-modified cells produced according to the methods of the invention for any number of human or veterinary therapeutics. For example, vector types other than above mentioned combination of genes can be produced as described herein can be used in ex vivo cell transplantation therapies for the treatment of a variety of human diseases, e.g., disorders of the immune system. Accordingly, such cells are useful for modulating autoimmunity and limiting a variety of autoimmune diseases.

Gene-modified cancer cells also find use in the ex vivo expansion of T cells, e.g., CD4⁺ cells or CD8⁺ cells or both. Thus, such cells are useful for stimulating the proliferation and reconstitution of CD4⁺ cells or CD8⁺ cells or both in a human having an immune disorder. Reconstitution of the immune system, e.g., a patient's CD4⁺ cells, is useful in immunotherapy for preventing, suppressing, or inhibiting a broad range of immunological disorders, e.g., as found during HIV infection.

The cancer cells described herein are also useful for vaccine development. For example, administration of antigens (as a form of cell lysate) to immuno-competent host further facilitate the use of these cells for active immunization in situ.

In addition, gene-modified cancer cells are useful for the generation of antibodies (e.g., monoclonal antibodies) that recognize cancer cell-specific markers. Anti-cancer cell antibodies are produced according to standard hybridoma technology. Such antibodies are useful for the evaluation and diagnosis of a variety of immunological disorders.

In some embodiments, the ACV as described herein at therapeutically effective concentrations or dosages may be combined with a pharmaceutically or pharmacologically acceptable carrier, excipient or diluent, either biodegradable or non-biodegradable. Exemplary examples of carriers include, but are by no means limited to, for example, poly(ethylene-vinyl acetate), copolymers of lactic acid and glycolic acid, poly(lactic acid), gelatin, collagen matrices, polysaccharides, poly(D,L lactide), poly(malic acid), poly(caprolactone), celluloses, albumin, starch, casein, dextran, polyesters, ethanol, mathacrylate, polyurethane, polyethylene, vinyl polymers, glycols, mixtures thereof and the like. Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars and starches. See, for example, Remington: The Science and Practice of Pharmacy, 2000, Gennaro, AR ed., Eaton, Pa.: Mack Publishing Co.

The invention provides kits for carrying out the methods of the invention. Accordingly, a variety of kits are provided. The kits may be used for any one or more of the following (and, accordingly, may contain instructions for any one or more of the following uses): treating some forms of cancer in an individual; preventing the spread or metastasis of some forms of cancer; preventing one or more symptoms of some forms of cancer; reducing severity of one or more symptoms associated with cancer; delaying development of cancer in an individual; or vaccinating an individual against some forms of cancer.

The kits of the invention comprise one or more containers comprising the ACV and a suitable excipient as described herein and a set of instructions, generally written instructions although electronic storage media (e.g., magnetic diskette or optical disk) containing instructions are also acceptable, relating to the use and dosage of the ACV for the intended treatment. The instructions included with the kit generally include information as to dosage, dosing schedule, and route of administration for the intended treatment. The containers of the ACV may be unit doses, bulk packages (e.g., multi-dose packages) or sub-unit doses.

The ACV may be packaged in any convenient, appropriate packaging.

As will be appreciated by one knowledgeable in the art, the anti-cancer vaccine may be combined or used in combination with other treatments known in the art.

While the preferred embodiments of the invention have been described above, it will be recognized and understood that various modifications may be made therein, and the appended claims are intended to cover all such modifications which may fall within the spirit and scope of the invention.

1. A method of eliciting an anti-tumor immune response in a patient comprising:
isolating cancerous cells from a patient;
transfecting said cancerous cells with an expression vector system comprising a DNA molecule encoding IL-12 and a costimulatory molecule operably linked to a promoter capable of directing expression of said DNA molecule in said cancerous cells;
incubating said transfected cells under conditions whereby the IL-12 and the costimulatory molecules are expressed; and
eliciting an anti-tumor immune response in the patient by injecting said transfected cells into the patient.

2. The method according to claim 1 including irradiating the cancerous cells to prevent replication of the cancerous cells prior to injection of the cancerous cells into the patient.

3. The method according to claim 1 wherein IL-12 is a single gene fusion of p35 and p40.

4. The method according to claim 1 wherein the costimulatory molecule is B7-1.

5. A method of vaccinating an individual comprising:
providing cancerous cells isolated from a donor;
transfecting said cancerous cells with an expression vector system comprising a DNA molecule encoding IL-12 and a costimulatory molecule operably linked to a promoter capable of directing expression of said DNA molecule in said cancerous cells;
incubating said transfected cells under conditions whereby the IL-12 and the costimulatory molecules are expressed;
isolating naive T cells from the individual;
exposing the T cells to the transfected cancerous cells, thereby activating the T cells;
separating the active T cells from the transfected cancerous cells; and
injecting the activated T cells into the patient.

6. The method according to claim 5 wherein IL-12 is a single gene fusion of p35 and p40.

7. The method according to claim 5 wherein the costimulatory molecule is B7-1.

8. An expression system comprising a DNA molecule encoding IL-12 and a costimulatory molecule operably linked to a promoter.

9. The expression system according to claim 8 wherein the IL-12 comprises a fusion of p35 and p40.

10. The expression system according to claim 8 wherein the costimulatory molecule is selected from the group consisting of: B7-1; B7-2 and CD40L.

11. The expression system according to claim 8 wherein the costimulatory molecule is B7-1.

12. A cancerous cell transfected with the expression system of claim 8.

13. An anti-cancer vaccine comprising the cancerous cell of claim 12 and a suitable excipient.
